(19) United States
(12) Patent Application Publication
(10) Pub. No.: US 2025/0297977 A1
(43) Pub. Date: Sep. 25, 2025

(54) SENSOR ELEMENT AND GAS SENSOR

(71) Applicant: TDK CORPORATION, Tokyo (JP)

(72) Inventors: Takahiro HAMAMURA, Hong Kong (CN); Yong Bing Hu, Hong Kong (CN)

(21) Appl. No.: 19/078,505

(22) Filed: Mar. 13, 2025

(30) Foreign Application Priority Data
Mar. 22, 2024 (CN) 202410332825.7

(51) Int. Cl. G01N 25/18; G01N 33/00

(52) U.S. Cl. CPC G01N 25/18; G01N 33/0027

(57) ABSTRACT

The disclosure discloses a sensor element and a gas sensor, comprising a substrate and a thin film arranged on the substrate; The substrate is provided with a cavity and an opening in communication with the cavity; The thin film is supported on the opening and partially covers the cavity; The thin film comprises first and second heating resistors, each of which is shaped in a meander line, the first heating resistor is positioned on a side of the thin film near the cavity, and the second heating resistor is positioned on a side of the thin film away from the cavity; and a linear segment at an end portion of the first heating resistor is at least partially overlapped with a linear segment at an end portion of the second heating resistor, to allow the thermal stresses to cancel out each other.

### CROSS-REFERENCE TO RELATED APPLICATIONS

This application claims priority to CN 202410332825.7 filed Mar. 22, 2024, the entire contents of which are hereby incorporated by reference.

### TECHNICAL FIELD

The present disclosure relates to the technical field of gas sensors, more particularly to a sensor element and a gas sensor.

### BACKGROUND

The gas sensor is a device for sensing gases and their concentrations in an environment. It converts the information related to types and concentrations of gases into electrical signals for detection, monitoring, analysis, and alarm. The gas sensor is equipped with a heating element and a film structure on the inside. The heating element can improve response of the gas sensor to gases and increase detection sensitivity. The film structure can inhibit heat transfer to the exterior, thereby ensuring that heating is effective only within the film and reducing energy required for heating.

However, during prolonged periods of use, the heat energy generated by the heating inside the gas sensor would cause thermal stresses, which may lead to a deformation of the film structure and thus a change in resistance characteristic inside the gas sensor, thereby affecting detection sensitivity. Therefore, it is urgent for those skilled in the art to address the technical problem of how to ensure a stable operation of the gas sensor and avoid deformation of the film structure.

### SUMMARY

The disclosure provides a sensor element and a gas sensor, with the design of a double-layer structure including a first heating resistor and a second heating resistor cooperated with each other, to enable the thermal stresses generated by heating to cancel out each other, thereby avoiding deformation of the film structure and ensuring stability of the gas sensor.

In order to solve the above-mentioned technical problems, an aspect of the disclosure provides a sensor element comprising a substrate and a thin film arranged on the substrate;

The substrate is provided with a cavity and an opening in communication with the cavity;

The thin film is supported on the opening and partially covers the cavity;

The thin film comprises a first heating resistor and a second heating resistor, each of the first heating resistor and the second heating resistor is shaped in a meander line, the first heating resistor is positioned on a side of the thin film near the cavity, and the second heating resistor is positioned on a side of the thin film away from the cavity;

A linear segment at an end portion of the first heating resistor is at least partially overlapped with a linear segment at an end portion of the second heating resistor, to allow a thermal stress induced by the first heating resistor and a thermal stress induced by the second heating resistor to cancel out each other.

In a preferred example, the thin film may further comprise a thermistor electrode and a thermosensitive resistor material;

The thermistor electrode may lie on a same plane as either the first heating resistor or the second heating resistor, and the thermosensitive resistor material may at least partially cover the thermistor electrode.

In a preferred example, the thin film may further comprise a thermistor electrode and a thermosensitive resistor material;

The first heating resistor may be positioned on a side of the thin film near the cavity;

The second heating resistor may be positioned on a side of the thin film away from the first heating resistor;

The thermistor electrode may be positioned on a side of thin film away from the second heating resistor, and the thermosensitive resistor material may at least partially cover the thermistor electrode.

In a preferred example, the thin film may further comprise a first thermistor electrode, a first thermosensitive resistor material and a second thermistor electrode;

The first thermistor electrode may lie on a same plane as the first heating resistor, the first thermosensitive resistor material may at least partially cover the first thermistor electrode, and the second thermistor electrode may lie on a same plane as the second heating resistor.

In a preferred example, the thin film may further comprise the second thermosensitive resistor material, and the second thermosensitive resistor material may at least partially cover the second thermistor electrode.

In a preferred example, the thermistor electrode may be disposed inside a non-linear portion of the first heating resistor or the second heating resistor.

In a preferred example, the non-linear portion of the first heating resistor may be curved in a direction rotated 180° relative to a direction in which the non-linear portion of the second heating resistor is curved.

In a preferred example, the first heating resistor and the second heating resistor may be connected in parallel in a circuit.

In a preferred example, the circuit may be further provided with a voltage amplifier for adjusting a voltage applied to the first heating resistor and the second heating resistor.

In a preferred example, wherein the thin film may further comprise an insulator provided between the first heating resistor and the second heating resistor, and the first heating resistor and the second heating resistor may be isolated from each other by the insulator.

In a preferred example, wherein the thin film may further comprise a first insulator;

The thermistor electrode may lie on a same plane as the second heating resistor, and the thermosensitive resistor material may at least partially cover the thermistor electrode and the second heating resistor;

The first insulator may be positioned on a layer that is lower than the second heating resistor; the first insulator may partially cover the first heating resistor, and the first heating resistor may be positioned on a side of the first insulator away from the second heating resistor.

In a preferred example, the sensor element may further comprise a second insulator;

The second insulator may be positioned on a layer that is lower than the first heating resistor.

In a preferred example, the thin film may be provided with a dummy pattern for receiving heat energy to expand, to allow the deformation caused by the thermistor electrode to be eliminated.

In a preferred example, the sensor element may be provided with one or more thermal vias for allowing the first heating resistor and the second heating resistor to achieve thermal coupling.

In a preferred example, during operation of the sensor, a voltage applied to the first heating resistor may be a first voltage, and a voltage applied to the second heating resistor may be a second voltage, wherein the first voltage may be greater than or equal to the second voltage.

Another aspect of the disclosure provides a gas sensor comprising the sensor element as mentioned above.

Compared with prior arts, the embodiments of the disclosure have advantages including at least one of the following.

The first heating resistor and the second heating resistor can cooperate with each other. The thermal stress in the thickness direction of the film caused by the heat generated by the first heating resistor can be opposite in direction to the thermal stress in the thickness direction of the film caused by the heat generated by the second heating resistor. This can allow the stresses to cancel out each other, and thus cancel out the thermal stress in the thickness direction of the film. In such a case, the thermal stress in the thickness direction of the film can be significantly reduced, thereby effectively avoiding deformation of the film structure, stabilizing the resistance characteristics within the gas sensor, and making them less prone to change. Consequently, the stable performance of the gas sensor can be ensured.

### BRIEF DESCRIPTION OF THE DRAWINGS

FIG. 1 is a structural schematic view according to a first embodiment of the disclosure;

FIG. 2 is a side view, taken along x-y plane, according to the first embodiment of the disclosure;

FIG. 3 is a top view, taken along a plane Ain FIG. 2, according to the first embodiment of the disclosure;

FIG. 4 is a schematic view taken along a plane Bin FIG. 2, viewed from top, according to the first embodiment of the disclosure;

FIG. 5 is a schematic diagram of a circuit for driving a first heating resistor and a second heating resistor of the disclosure;

FIG. 6 illustrates a simulation result when a voltage is applied to a first heating resistor;

FIG. 7 illustrates a simulation result when a voltage is applied to a second heating resistor;

FIG. 8 illustrates a simulation result when voltages are applied to both a first heating resistor and a second heating resistor according to the first embodiment;

FIG. 9 is a schematic view illustrating a comparison of simulation results of FIGS. 6-8 of the disclosure;

FIG. 10 is a structural schematic view according to a second embodiment of the disclosure;

FIG. 11 is a side view (in a direction along axis −y) according to the second embodiment of the disclosure;

FIG. 12 is a top view (in a direction along axis +z) taken along a plane A in FIG. 11 of the disclosure;

FIG. 13 is a top view (in a direction along axis +z) taken along a plane B in FIG. 11 of the disclosure;

FIG. 14 is a structural schematic view according to a third embodiment of the disclosure;

FIG. 15 is a side view (in a direction along axis −y) according to the third embodiment of the disclosure;

FIG. 16 is a top view (in a direction along axis +z) taken along a plane A in FIG. 15 of the disclosure;

FIG. 17 is a view in a direction (along +z) taken along a plane B in FIG. 15 of the disclosure;

FIG. 18 is a schematic diagram illustrating two thermistor electrodes according to the third embodiment of the disclosure;

FIG. 19 is a structural schematic view according to a fourth embodiment of the disclosure;

FIG. 20 is a side view (in a direction along axis-y) according to the fourth embodiment of the disclosure;

FIG. 21 is a schematic view of a plane A, viewed from top, according to the fourth embodiment of the disclosure;

FIG. 22 is a schematic view of a plane B, viewed from top, according to the fourth embodiment of the disclosure;

FIG. 23 is a structural schematic view according to a fifth embodiment of the disclosure;

FIG. 24 is a plane view according to the fifth embodiment of the disclosure, corresponding to FIG. 4 of the first embodiment;

FIG. 25 is a schematic diagram of a circuit for driving a first heating resistor and a second heating resistor of the disclosure;

FIG. 26 is a schematic diagram of a circuit with a voltage amplifier of the disclosure;

FIG. 27 illustrates a first shape structural design of individual parts of the disclosure;

FIG. 28 illustrates a second shape structural design of individual parts of the disclosure;

FIG. 29 illustrates a third shape structural design of individual parts of the disclosure;

FIG. 30 illustrates a fourth shape structural design of individual parts of the disclosure;

FIG. 31 illustrates a fifth shape structural design of individual parts of the disclosure;

FIG. 32 illustrates a sixth shape structural design of individual parts of the disclosure;

FIG. 33 is a schematic plane view of the disclosure, provided with a first virtual pattern structure;

FIG. 34 is a schematic plane view of the disclosure, provided with a second virtual pattern structure;

FIG. 35 is a schematic plane view of the disclosure, provided with a third virtual pattern structure;

FIG. 36 is a schematic plane view of the disclosure, provided with a fourth virtual pattern structure;

FIG. 37 is a schematic plane view of the disclosure, provided with a fifth virtual pattern structure;

FIG. 38 shows relevant curve graphs of the disclosure;

### REFERENCE NUMERALS

Herein, 101. sensor element; 102. substrate; 103. cavity; 104. thin film; 105. first heating resistor; 105a. end of first heating resistor; 105n. end of first heating resistor; 106. second heating resistor; 106a. end of second heating resistor; 106n. end of second heating resistor; 107. thermistor electrode; 108. thermosensitive resistor material; 109a. bonding wires; 109b. bonding wires; 109c. bonding wires; 109d. bonding wires; 110. insulators; 110a. first insulator; 110b. second insulator; 111. contact surface between first heating resistor and connection pads; 112. gaps; 113a. connection pads; 113d. connection pads; 114.

virtual pattern (dummy pattern); 115. second thermistor electrode; 116. thermal vias; 201. the voltage source; 202. resistance of first heating resistor; 203. resistance of second heating resistor; 204. series circuit; 205. resistance of fourth heating electrode; 206. resistance of fifth heating electrode; 207. resistance of thermistor electrode; 208. resistance of second thermistor electrode.

### DETAILED DESCRIPTION OF ILLUSTRATED EMBODIMENTS

The technical solutions according to the embodiments of the present disclosure will be clearly and completely explained below in conjunction with the drawings for the embodiments of the present disclosure. Apparently, the described embodiments are merely a part rather than all of the embodiments of the present disclosure, and they are provided in order to facilitate thorough and comprehensive understanding of the present disclosure. All embodiments obtained by those skilled in the art based on the embodiments of the present disclosure without creative work shall fall within the scope of the present disclosure.

The terms such as “first”, “second”, and “third” used in the description are used for convenience of description and are not intended to indicate or imply relative importance or hint the quantity of components. Hence, features defined by the terms “first”, “second”, and “third” are intended to indicate or hint one or more of such features. Unless explicitly stated otherwise, “the plurality of” as used in the description refers to two or more.

It should be noted that, unless explicitly defined or specified otherwise, terms such as “mount”, “connect” and “attach” used in the description are intended to have meanings understood in a broad sense. For example, “connect” may refer to fixedly connect, or detachably connect, or integrally connect; or mechanically connect, or electrically connect; or directly connect, or indirectly connect via an intermedium, or internally communicate two components. The terms, such as “vertical”, “horizontal”, “left”, “right”, “upper”, “lower”, and the like, are used in the description for purposes of illustration rather than indicating or hinting a limitation in terms of specific orientation or configuration and operation with specific orientation to the described device or element, and should not be regarded as a limitation to the present disclosure. The term “and/or” as used in the description is intended to cover any one or a plurality of so-described items and all combinations. Those skilled in the art can understand specific meanings of the aforementioned terms used herein in accordance with specific conditions.

It should be noted that, unless otherwise defined in the description of the disclosure, all technical and scientific terms used herein have same meanings as commonly understood by those skilled in the art. The terms used in the description of the disclosure are merely for the purpose of illustrating specific embodiments and are not intended to limit the invention. Those skilled in the art can understand specific meanings of the aforementioned terms used herein in accordance with specific conditions.

### First Embodiment

It should be noted beforehand that the heating components/devices within the gas sensor are usually designed with planar pattern coils (such as meandering or spiral coils). The planar coils which are relatively thin can effectively heat the planar film structure. Concerning the film structures for achieving heating, a film structure with a relatively greater thickness would have a relatively higher thermal capacity, and thus more energy needs to be input for heating, which may lead to uneven heating within the film. Hence, it is necessary to ensure that the film structures have a relatively small thickness. However, in such a case that a film structure has a very small thickness, the heat energy generated during heating would cause thermal stresses, thereby leading to a deformation of the film structure. Moreover, such deformation would be exacerbated during prolonged use of the gas sensor.

In heating-type gas sensors, the thermistor is usually mounted on the upper surface or lower surface of a film that is in contact with the external air. Deformation of the film would cause a structural deformation of the thermistor, thereby leading to a change in its resistance characteristics. Hence, in order to provide a gas sensor with stable characteristics in a long time, it is crucial to prevent deformation of the thin film.

According to the first embodiment of the disclosure, a sensor element 101 is provided. Please referring to FIG. 1 which shows a structural schematic view according to a first embodiment of the disclosure, it particularly includes a substrate 102 and a thin film 104 disposed on the substrate 102. The thin film 104 comprises a first heating resistor 105 and a second heating resistor 106.

The substrate 102 is also known as the base material, which needs to possess an adequate mechanical strength. Without any particular limitation, it may be a material suitable for micro-processing such as etching. For example, it may be a silicon monocrystalline substrate, a sapphire monocrystalline substrate, a ceramic substrate, a quartz substrate, a glass substrate, etc., which is not specifically defined herein.

In the embodiment of the disclosure, the substrate 102 has a rectangular prism structure, with a cavity 103 being provided in its center and an opening for communicating with the cavity 103 being provided on the surface of the substrate 102. The thin film 104 is disposed above the substrate 102. Due to the presence of the cavity 103, the central portion of the thin film 104 is not in direct contact with the substrate 102. The abovementioned opening is partially covered by the thin film 104. As shown in FIG. 1, the thin film 104 is disposed on the central portion of the opening and is supported above the opening by four diagonal arms. As the opening is not completely covered by the thin film 104, gaps 112 (i.e., the four trapezoid-like gaps) are further provided, as shown in FIG. 1. With such arrangement, the thin film 104 can be supported at several points around the opening and can be fixed on the substrate 102.

The first heating resistor 105 and the second heating resistor 106 both consist of the meander line, which includes a linear straight segment and a nonlinear meandering segment. For example, the input/output line of the meander line (meandering shape) of the first heating resistor 105 at the lower layer is located under the input/output line of the meander line (meandering shape) of the second heating resistor 106 at the upper layer. The straight segments at the beginning and the end of the meandering segment of the first heating resistor 105 are at least partially overlapped with the straight segments at the beginning and the end of the meandering segment of the second heating resistor 106. Preferably, the first heating resistor 105 and the second heating resistor 106 are isolated by insulators 110 to prevent electrical contact. Additionally, the substrate 102 may be made of a conductive material by MEMS processes, such as the well-known silicon substrate. In such a case, the substrate 102 and the first heating resistor 105 are isolated by the insulators 110 to prevent electrical contact therebetween.

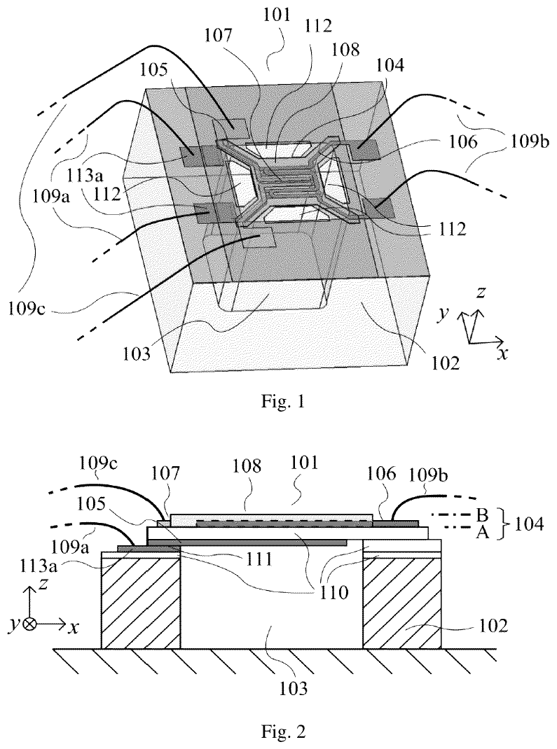

In the embodiment, the first heating resistor 105 is located below the thin film 104, while the second heating resistor 106 is located above the thin film 104. Referring to FIG. 2 which shows a side view according to the first embodiment of the disclosure on the x-y plane, the first heating resistor 105 is in electrical contact with the connection pads 113a through the contact surface 111 between the first heating resistor and the connection pads, and the bonding wires 109a are connected to the connection pads 113a which are in contact with the first heating resistor.

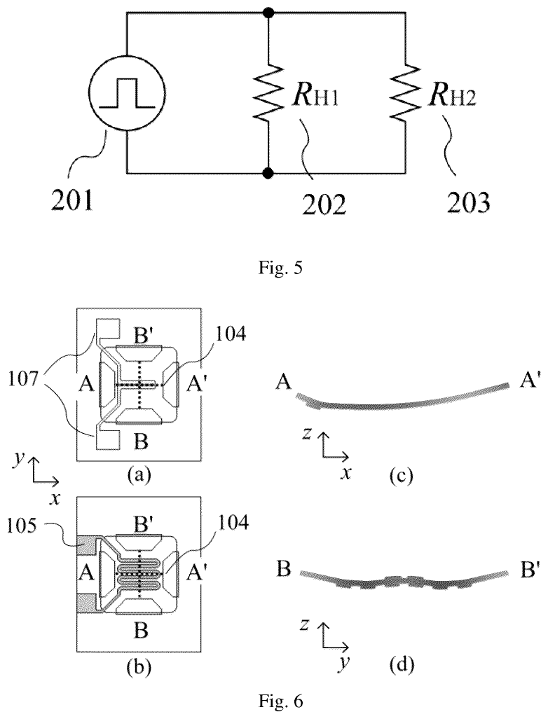

Similarly, two ends of the second heating resistor 106 are respectively correspondingly provided with a part in the shape of the abovementioned connection pad, and the bonding wires 109b are connected to the parts in the shape of the abovementioned connection pad, respectively. The ends of the bonding wires 109a and 109b are omitted in the drawings, however, these ends are connected to the circuits that supply power for the first heating resistor 105 and the second heating resistor 106, as shown in FIG. 5.

As mentioned above, the thin film 104 is connected with the substrate 102 through the cavity 103 or the gaps 112, to reduce the thermal capacity of the thin film 104 and meanwhile decrease the thermal conduction between the thin film 104 and the substrate 102. The reduction in the thermal capacity and the thermal conduction of the thin film 104 makes it possible for the thin film 104 to be heated efficiently and rapidly through the Joule heat generated by applying voltage to the first heating resistor 105 and the second heating resistor 106. Furthermore, when the voltages of the first heating resistor 105 and the second heating resistor 106 are changed to 0 (turned off), the temperature drops more quickly.

The abovementioned sensor element can form a compact gas sensor heating device. However, in order to constitute the gas sensor, it is also necessary to arrange a thermistor electrode 107 and a thermosensitive resistor material 108 on the thin film 104.

The thermistor electrode 107 is a pattern made of a conductor such as copper, silver, gold, or platinum. The thermistor electrode 107 lies on the same plane as either the first heating resistor 105 or the second heating resistor 106. In the aforementioned embodiment, the thermistor electrode 107 is arranged on the same plane as the second heating resistor 106. As shown in FIGS. 1 and 4 (FIG. 4 shows a schematic view of the plane B viewed from top according to the first embodiment as depicted in FIG. 2 of the disclosure, in which the first heating resistor and the bonding wires connected to the first heating resistor are omitted to facilitate understanding of the configuration at the top), the thermistor electrode 107 is preferably disposed in the groove defined by the non-linear portion (meandering shape) of the second heating resistor 106. Due to the thermistor electrode 107 arranged in this way, heat transfer efficiency can be improved, and the Joule heat generated by the second heating resistor 106 can be obtained more effectively.

Referring to FIGS. 1 and 4, the thermosensitive resistor material 108 is arranged to be in contact with the thermistor electrode 107, and the thermistor electrode 107 is at least partially covered by the thermosensitive resistor material 108.

The thermosensitive resistor material 108 is a material with a resistance varying with temperature. When the thin film 104 is heated by the first heating resistor 105 or the second heating resistor 106, the temperature rises and thus the resistance of the thermosensitive resistor material 108 changes. Since the thermosensitive resistor material 108 is in contact with the thermistor electrode 107, the resistance of the thermistor electrode 107 changes as the resistance of the thermosensitive resistor material 108 changes.

In FIG. 4, the thermistor electrode 107 preferably consists of two patterns 107a and 107b. In order to enable the resistance values between the thermistors to reach the order of kΩ or higher, two or more patterns may be used, and furthermore, they may be arranged with a certain gap between their surfaces. But alternatively, a single pattern may be used, without defining a gap, if the resistance to be provided is of an order of magnitude lower than that of 100 ohms.

Generally speaking, the gas-heated type of gas sensors can improve accuracy by heating the gas through heating electrodes. The thermal conductivity gas sensors can determine the gas concentration based on temperature changes during the gas heating process, and measure concentration by utilizing the changes in the thermal conductivity of the gas during the heating process. When the gas is heated by the heating electrodes, the thermistors can detect temperature changes, so as to achieve determination of the gas concentration. The thermistors are commonly used for such type of temperature detection.

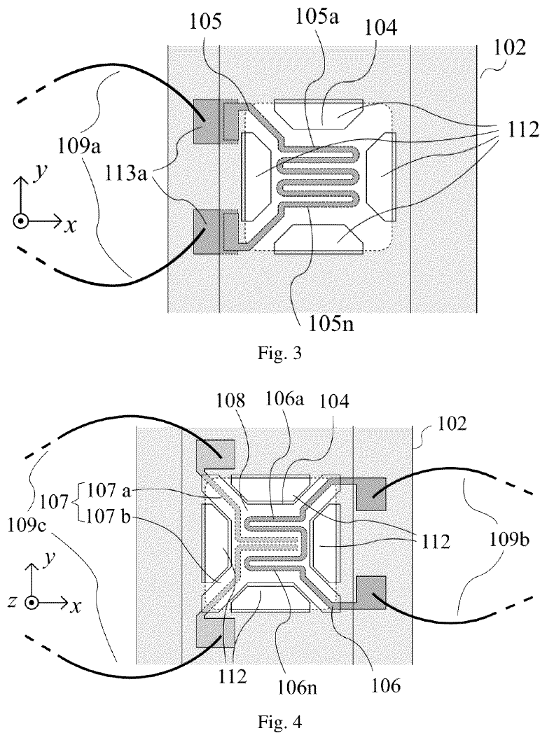

As shown in FIG. 4, the thermistor electrode 107 and the second heating resistor 106 are arranged on the same plane. As mentioned above, the heating can be performed by the second heating resistor 106, or performed through thermal conduction by the first heating resistor 105 on the lower plane as depicted in FIG. 3 (FIG. 3 shows a top view of the plane Ain FIG. 2 according to the first embodiment of the disclosure). Consequently, the method for effectively heating the thermistor electrode 107 may be selected according to actual needs.

Furthermore, the thermosensitive resistor material 108 may be arranged to cover only the thermistor electrode 107, or alternatively, to further cover the second heating resistor 106 as well, to facilitate manufacturing. Apparently, it may cover the entire thin film 104, as shown in FIGS. 1 and 4. One skilled in the art can make a selection based on actual process requirements.

As shown in FIGS. 1-4, the bonding wires 109a, 109b, and 109c, are wires made of materials such as gold, aluminum, and copper. They are connected with the connection pads 113a which are in connection with the first heating resistor 105, the connection pads which are disposed at two ends of the second heating resistor 106, and the connection pads which are disposed at two ends of the thermistor electrode 107, by using heat, ultrasound, or pressure.

Furthermore, the other ends of the bonding wires 109a and 109b are connected to a circuit for applying a heating voltage, while the other ends of the bonding wires 109c are connected to a circuit for detecting changes in the resistances of the thermistors.

FIG. 5 shows an example circuit for driving the first heating resistor 105 and the second heating resistor 106. The figure shows resistors 202 and 203 which are connected in parallel with the voltage source 201. The resistor 202 is equivalent to the resistances of the first heating resistor 105 and the bonding wires 109a. In other words, it illustrates how the ends of the bonding wires 109a, which are not connected to the connection pads 113a in FIGS. 1 to 3, are connected to the circuit of FIG. 5. On the other hand, the resistor 203 is equivalent to the resistances of the second heating resistor 106 and the bonding wires 109b. In other words, it illustrates how the other ends, of the bonding wires 109b which are in contact with the connection pads on both ends of the second heating resistor 106 in FIGS. 1, 2, and 4, are connected to the circuit of FIG. 5.

That is, in FIG. 5, the resistance 202 of the first heating resistor 105 is indicated by RH1, and the resistance 203 of the second heating resistor 106 is indicated by RH2. In the first embodiment, RH1 is greater than RH2, which is determined by their shapes and sizes, specifically as illustrated in FIGS. 3 and 4, where the length of the pattern of the first heating resistor 105 is longer than that of the second heating resistor 106.

In the circuit of FIG. 5, the resistance 202 and the resistance 203 are connected in parallel and then connected to the voltage source 201. Hence, when the voltage source 201 generates a voltage, it simultaneously applies the voltage to both the resistance 202 and the resistance 203.

In other words, the voltage generated by the voltage source 201 is applied to both the first heating resistor 105 and the second heating resistor 106 at the same time, allowing both the first heating resistor 105 and the second heating resistor 106 to consume power and generate heat.

As the circuit in FIG. 5 includes neither capacitive elements nor inductive elements, the time to generate Joule heat by the first heating resistor 105 and the second heating resistor 106 is the same. Besides, the voltage source 201 is depicted as a simple pulse source symbol in FIG. 5, however, the voltage level which can vary over time may be a sine wave, triangular wave, or PWM wave. It is crucial that the driving time of the first heating resistor 105 and the second heating resistor 106 need to be the same.

To illustrate the derivation of the disclosure in detail, the simulation experiments are separately described as follows.

FIG. 6 shows the simulation result when a voltage is applied to the first heating resistor. FIG. 6 shows the simulation result in such a case that the thermistor 107 and the first heating resistor 105 are arranged on the thin film 104. As mentioned above, the first heating resistor 105 is disposed on the lower surface of the film 104, while the thermistor electrode 107 is disposed on the upper surface of the film 104. In such a case, during operation, the sensor element undergoes a deformation as illustrated on the right (bending downwards).

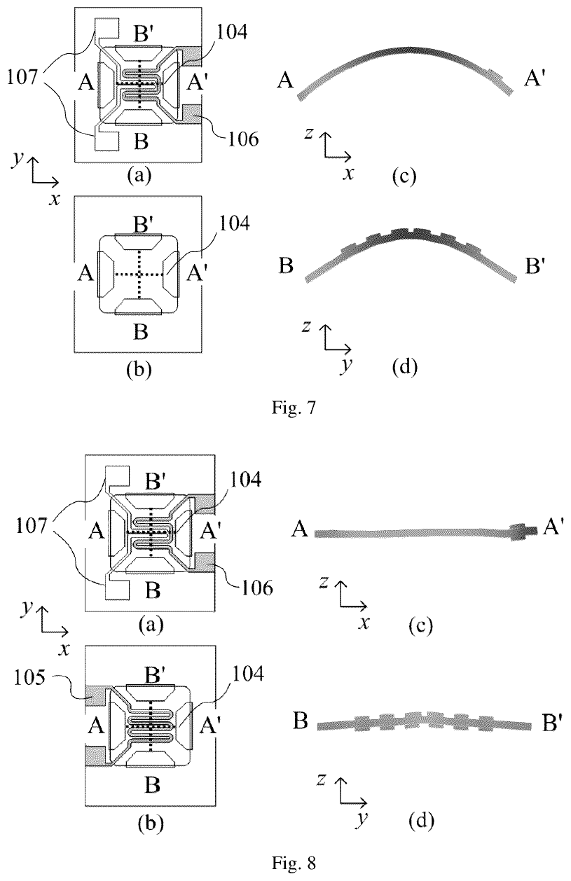

FIG. 7 shows the simulation result when a voltage is applied to the second heating resistor. FIG. 7 shows the simulation result in such a case that the thermistor 107 and the second heating resistor 106 are arranged on the thin film 104. As mentioned above, the second heating resistor 106 and the thermistor 107 are disposed on the upper surface of the film 104. In such a case, during operation, the sensor element undergoes a deformation as illustrated on the right (bending upwards).

Accordingly, with opposite bending directions, the dual-layer of the heating resistors can provide an appropriate design to allow the upward and downward deformations to cancel out each other. FIG. 8 shows the simulation result when the voltages are applied to both the first heating resistor and the second heating resistor according to the first embodiment. That is, the first heating resistor 105 is disposed on the lower surface of the film 104, while the second heating resistor 106 and the thermistor electrode 107 are disposed on the upper surface of the film 104. In such a case, the deformation is as illustrated on the right, with almost no obvious bending.

During actual analysis, the simulation can be performed based on the finite element method, by employing electro-thermal-thermal stress coupling analysis. The electrical analysis can be conducted first. During the electrical analysis, the current values and the resistance values of each component can be calculated based on the voltage applied to the connection pads, and the resistivity and shape parameters of the heating resistors, the thermistor electrode, the thermosensitive resistor material, the insulators, and the substrate materials. Subsequently, the thermal analysis can be performed. During the thermal analysis, Joule heat can be calculated based on the current values and the resistance values obtained from the electrical analysis, and the temperatures of individual parts can be calculated based on the Joule heat and the thermal conductivity, the heat transfer coefficient, the thermal radiation coefficient, and the specific heat. Herein, when conducting the electrical analysis again, the changes in resistivities caused by the temperature increase should be considered, especially for metal components that have resistivities changing significantly with the temperature increases. The values of the current flowing there through and the resistances of individual parts can be calculated based on the resistivity and shape parameters updated according to temperature increases. In this way, the values of the resistivities, the currents, the Joule heats, and the temperatures of individual parts can be calculated by repeated electrical and thermal analyses upon convergence. Finally, the thermal stress analysis can be further conducted. The thermal stress can be calculated based on the temperatures and the linear expansion coefficient, Young's modulus, and Poisson's ratio of individual parts. Furthermore, the strains of individual parts can be calculated.

It should be noted that, in order to make the deformations in the drawings clear so as to facilitate understanding, the effects of bending deformations have been illustrated in the simulation graphs as shown in FIGS. 6 to 8 in an enlarged and exaggerated manner.

In addition, since too complicated simulation models would require excessive memory for analysis and bring difficulties to analysis, the connection pads 113a have been omitted from the simulation model. For the same reason, the thermosensitive resistor material 108 has also been omitted from the simulation. FIGS. 6(a) and 6(b) show views of the thin film 104, viewed from the top. FIG. 6(a) shows the pattern on the upper surface of the thin film 104, i.e., showing the thermistor electrode 107 on the upper surface of the thin film 104, but not showing the first heating resistor 105 on the lower surface of the thin film 104. On the other hand, FIG. 6(b) merely shows the pattern on the bottom surface of the thin film 104, i.e., it merely shows the first heating resistor 105.

FIGS. 6(c) and 6(d) show one of the simulation results when a voltage is applied to the first heating resistor 105. FIG. 6(c) illustrates the deformation at the cross-section A-A′ of FIGS. 6(a) and 6(b). FIG. 6(d) further illustrates the deformation at the cross-section B-B′ of FIGS. 6(a) and 6(b).

These results indicate that when the meandering-shaped heating resistor is only provided on the bottom surface of the thin film 104, the thin film 104 undergoes a relatively great deformation downwards (in the −z direction). This is because the first heating resistor 105, which is heated to expand, causes deformation of the thin film 104. Therefore, when the first heating resistor 105 on the bottom of the thin film 104 undergoes the thermal expansion, the thin film 104 itself deforms in the −z direction.

FIG. 7 shows the simulation result when the heating resistor is only provided on the upper surface of the thin film 104. FIGS. 7(a) and 7(b) show top views of the thin film 104. Herein, FIG. 7(a) merely illustrates the pattern on the upper surface of the thin film 104. That is, it illustrates both the thermistor electrode 107 and the second heating resistor 106 on the upper surface of the thin film 104. On the other hand, FIG. 7(b) illustrates the pattern on the bottom surface of the thin film 104. However, there is no pattern on the bottom surface of the thin film 104 according to the arrangement of FIG. 7, thus no further description is provided.

FIGS. 7(c) and 7(d) are schematic views which illustrate the deformations of the thin film 104. They illustrate the simulation results when a voltage is applied to the second heating resistor 106. FIG. 7(c) illustrates the deformation at the cross-section A-A′ of FIGS. 7(a) and 7(b). FIG. 7(d) further illustrates the deformation at the cross-section B-B′ of FIGS. 7(a) and 7(b). These results indicate that when the meandering-shaped heating resistor are merely provided on the upper surface of the thin film 104, the thin film 104 undergoes a relatively great deformation upwards (in the +z direction). This is because the second heating resistor 106, which is heated to expand, causes deformation of the thin film 104. Therefore, when the second heating resistor 106 on the upper surface of the thin film 104 undergoes the thermal expansion, the thin film 104 itself deforms in the +z direction.

From the comparison of FIG. 6 and FIG. 7, it can be seen that the meandering-shaped heating resistor is disposed on the bottom surface of the thin film 104 in the arrangement of FIG. 6, while the meandering-shaped heating resistor is disposed on the top surface of the thin film 104 in the arrangement of FIG. 7, which results in different deformation directions of the thin film 104. This is because the significant expansion of the heating resistors in the meandering longitudinal direction is the primary cause of the deformation of the thin film 104.

Then, based on the first embodiment as depicted in FIGS. 1 to 4, the simulation results in such a case that the first heating resistor 105 and the second heating resistor 106 are arranged above and below the thin film 104 are illustrated by using FIG. 8. FIGS. 8(a) and 8(b) show top views of the thin film 104. Herein, FIG. 8(a) merely illustrates the pattern on the upper surface of the thin film 104. That is to say, it illustrates the thermistor electrode 107 and the second heating resistor 106 on the upper surface of the thin film 104. On the other hand, FIG. 8(b) illustrates the pattern on the bottom surface of the thin film 104. That is to say, it illustrates the first heating resistor 105 on the bottom surface of the thin film 104.

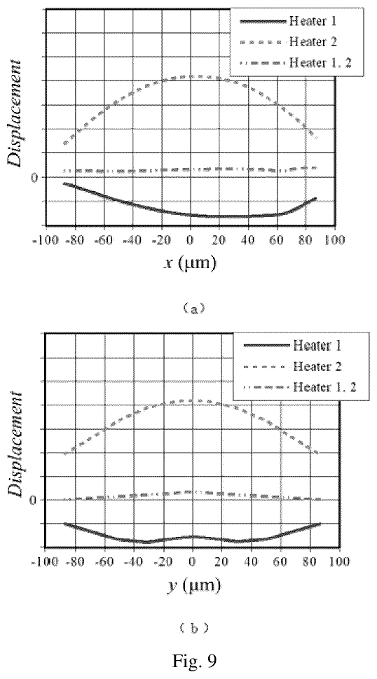

FIGS. 8(c) and 8(d) show one of the simulation results when voltages are applied to both the first heating resistor 105 and the second heating resistor 106. FIG. 8(c) illustrates the deformation at the cross-section A-A′ of FIGS. 8(a) and 8(b). FIG. 8(d) illustrates the deformation at the cross-section B-B′ of FIGS. 8(a) and 8(b). In FIG. 8, the overall result shows that the deformation of the thin film 104 is inhibited. To facilitate comparison of the results of FIGS. 6 to 8, a comparison chart is shown in FIG. 9, which illustrates a comparison of the simulation results of FIGS. 6 to 8.

FIG. 9 illustrates the changes of the top surface of the film according to the simulation results of FIGS. 6 to 8. FIG. 9(a) corresponds to FIGS. 6(c), 7(c), and 8(c), and show show it varies in the z-axis direction with the position of the film on the x-coordinate, using the film center x=0 as the x-coordinate.

FIG. 9(b) corresponds to FIGS. 6(d), 7(d), and 8(d), and show show it varies in the z-axis direction with the position of the film on the Y-coordinate, with the film center being y=0. The solid line in FIG. 9 is based on the simulation result of FIG. 6. One dashed line in FIG. 9 is drawn based on the simulation result of FIG. 7, with the same voltage being applied to facilitate comparison. In other words, in these simulations, the voltage applied to the first heating resistor 105 of FIG. 6 is the same as the voltage applied to the second heating resistor 106 of FIG. 7. Another dashed line in FIG. 9 shows the simulation result of FIG. 8, and similarly, the voltages applied to the first heating resistor 105 and the second heating resistor 106 of FIG. 8 are the same.

Apparently, FIG. 8 which simulates the voltages are applied to both the first heating resistor 105 and the second heating resistor 106 would consume more electrical energy and generate more heat. However, it has less deformation when compared to the configurations of FIGS. 6 and 7. This indicates that the first heating resistor 105 and the second heating resistor 106 tend to deform in opposite directions, thereby canceling out each other's displacements in the Z-axis direction. In other words, the configuration according to the first embodiment of the disclosure can significantly reduce the deformation of the thin film 104.

As mentioned above, if the thin film 104 is provided on its lower surface with the first heating resistor 105 and on its upper surface with the second heating resistor 106, when the voltage is applied to the first heating resistor 105 and the second heating resistor 106 simultaneously, the deformation of the thin film 104 in the Z-axis direction can be reduced, and the deformation in the Z-axis direction caused by long-term stress and heat can be inhibited. In this way, it can inhibit resistance changes of the thermistors resulted from the deformation, thereby ensuring sensor performance.

FIG. 8 shows the simulation result when voltages are applied to both the first heating resistor 105 and the second heating resistor 106 simultaneously. But alternatively, the voltage may be applied to either one of the first heating resistor 105 and the second heating resistor 106 to generate heat, if the thin film 104 is made of an extremely thin film. Once the heat is generated, the thermal flow may be transferred to the other heating resistor through the thin film, enabling simultaneous heating of both the first heating resistor 105 and the second heating resistor 106 to achieve simultaneous thermal expansion. Consequently, when the voltage is applied to only one of the heating resistors, both heating resistors can be heated to achieve the effect of cancelling out the displacements. In order to enable the first heating resistor 105 and the second heating resistor 106 to achieve a thermal coupling, transfer heat to each other and cancel out each other's thermal stress, the distance between them should be designed as small as possible. As the linear straight portion on the outermost side of the nonlinear meandering shape is particular useful in reducing the displacement of the thin film 104, the linear portion at the end of the first heating resistor 105 and the linear portion at the end of the second heating resistor 106 of the disclosure are at least partially overlapped, enabling the thermal stress induced by the first heating resistor 105 and the thermal stress induced by the second heating resistor 106 to cancel out each other.

Referring to FIGS. 3 and 4, the end 105a defined by a straight line and located at the beginning of the meandering shape of the first heating resistor 105, and the end 105n defined by a straight line and located at the end of the meandering shape of the first heating resistor 105 as shown in FIG. 3, and the end 106a defined by a straight line and located at the beginning of the meandering shape of the second heating resistor 106, and the end 106n defined by a straight line and located at the end of the meandering shape of the second heating resistor 106 as shown in FIG. 4, are preferably arranged above and below the thin film 104 in an overlapped manner. With such structure, the thermal coupling between the first heating resistor 105 and the second heating resistor 106 can be particularly strong at the outermost side of the meandering shape, such that the deformation can be effectively inhibited.

The first heating resistor 105 and the second heating resistor 106, among the components of the thin film 104, have arrangements and locations which are crucial for the reduction of deformation of the thin film 104 in the Z-axis direction, as the thermal expansions of the heating resistors caused by heating are greater than that of other components. For example, in the first embodiment, the meandering direction of the first heating resistor 105 and the meandering direction of the second heating resistor 106 may be at 180 degrees to each other by rotation. Though a configuration with their meandering directions being at 180 degrees to each other by rotation is provided in this embodiment, a configuration with their meandering directions being not at an angle to each other by rotation is preferred. In this way, most of the linear segments of the meandering shape can overlap above and below the thin film 104, thereby achieving enhanced deformation control.

As shown in FIGS. 3 and 4, the upper surface of the thin film 104 is provided with the thermistor 107, and the meandering shape of the first heating resistor 105 may extend to overlap under the thermistor 107. If the bottom of the thin film 104, where the thermistor 107 is positioned, is not provided with any pattern, the thermistor 107 would be the only one that undergoes the thermal expansion due to heating, easily leading to deformation of the thin film 104. However, if the first heating resistor 105 is further provided under the thermistor 107, the first heating resistor 105 will undergo the thermal expansion as well, thereby canceling out the deformation of the thermistor 107.

The above description focuses on the deformation caused by the thermal stress. From the perspective of effectively heating the thermistor, in the above configuration including the first heating resistor 105 and the second heating resistor 106, the heat generated by the first heating resistor 105 can be transferred below the thermistor, while the heat generated by the second heating resistor 106 which is arranged flat against the thermistor can be transferred from lateral sides of the thermistors, thereby allowing the thermistor to be heated effectively. According to the abovementioned configuration, not only is deformation of the thin film 104 caused by the thermal stress generated by the heating resistor can be reduced, but the configuration for effectively heating the thermistor can be also achieved.

### Second Embodiment

In the first embodiment, the first heating resistor 105 is disposed on the lower surface of the thin film 104, and the second heating resistor 106 is disposed on the upper surface of the thin film 104. Such configuration as depicted in the first embodiment requires an insulation treatment between the substrate 102 and the first heating resistor 105. Thus, it is required to provide a contact surface 111 for the first heating resistor 105 and the connection pads 113 and to bond the first heating resistor 105 and the connection pads 113 together, resulting in a complicated manufacturing process.

By providing an insulating layer under the first heating resistor 105, the manufacturing process of the first heating resistor 105 can become easier. In such a case, the first heating resistor 105 does not have to be arranged on the lower surface of the thin film 104. The present disclosure may also be applied to such structure.

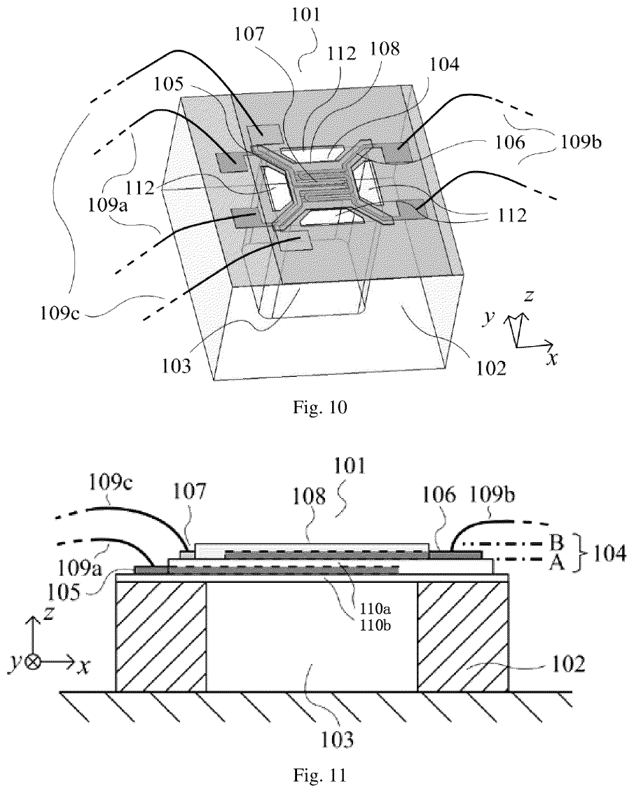

Please refer to FIGS. 10-13. FIG. 10 shows a structural schematic view according to a second embodiment of the disclosure. The first heating resistor 105 in the first embodiment is located at the bottom portion of the thin film 104, while in the second embodiment, the thin film comprises a thermosensitive resistor material 108, a thermistor electrode 107, a second heating resistor, and a first insulator. As shown in FIG. 11, the thermistor electrode 107 lies on the same plane as the second heating resistor 106, and the thermosensitive resistor material 108 at least partially covers the thermistor electrode 107 and the second heating resistor 106. The first insulator 110a is positioned on a layer that is lower than the second heating resistor 106. The first insulator 110a partially covers the first heating resistor 105, and the first heating resistor 105 is positioned on a side of the first insulator 110a away from the second heating resistor 106. Preferably, the sensor element may further comprise a second insulator 110b, and the second insulator 110b may be positioned on a layer that is lower than the first heating resistor 105.

Accordingly, the first heating resistor 105 is substantially sandwiched between the first insulator 110a and the second insulator 110b, thus the contact surface 111 between the first heating resistor 105 and the connection pads 113 can be omitted. The other arrangements of the second embodiment are identical to that of the first embodiment, and thus will not be repeated here.

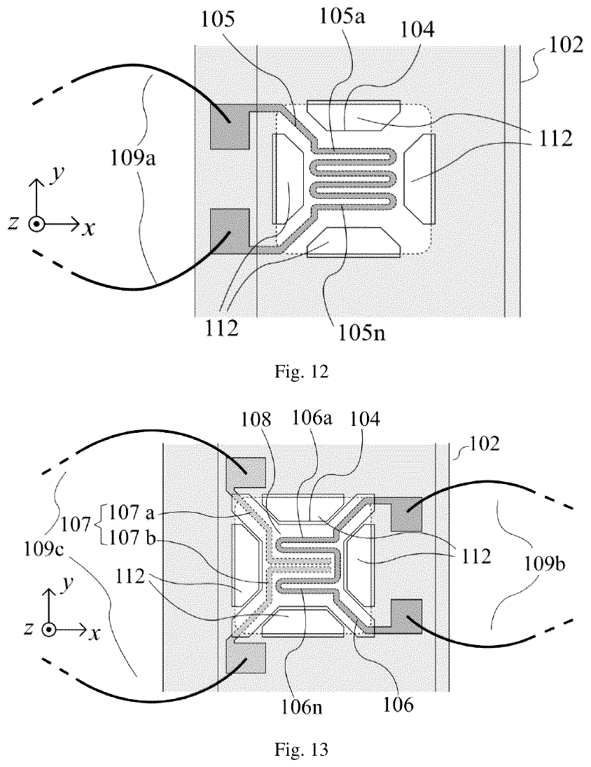

FIG. 11 shows a side view (in a direction along axis −y) according to the second embodiment of the disclosure. FIG. 12 shows a top view (in a direction along axis +z) taken along the plane A of FIG. 11. FIG. 13 shows a top view (in a direction along axis +z) taken along the plane B of FIG. 11. It should be noted that, the first heating resistor 105 arranged below the thin film 104 and the bonding wires 109a are omitted in FIG. 13, to facilitate a clear view of the arrangement of the top surface of the thin film 104. In such a case, similar to the first embodiment, if the first heating resistor 105 is located below the middle of the thickness of the film 104, when a voltage is applied to the first heating resistor 105, it will expand due to Joule heating, thereby resulting in the depression of the thin film 104 in the −z direction.

Hence, by applying a voltage to the second heating resistor 106 at the same time as the first heating resistor 105, the deformation of the thin film 104 can be cancelled out as in the first embodiment. Thus, as long as the first heating resistor 105 is positioned below the thin film 104 in its thickness direction, it does not necessarily need to be disposed on the lower surface of the thin film 104. The details will not be repeated here.

### Third Embodiment

The arrangement of the first heating resistor 105, second heating resistor 106, and the thermistor electrode 107 are described in the first and second embodiments. However, in order to cancel out the displacements in the Z-axis direction even more effectively, itis advantageous that the planar arrangement of the first heating resistor 105 and the second heating resistor 106 on the film 104 differs from the planar arrangement of the thermistor electrode 107. In another preferred implementation, the planar arrangement of the first heating resistor 105 on the thin film 104 may be the same as the planar arrangement of the second heating resistor 106. If the arrangements of the two planes are exactly the same, the Z-axis displacement can be eliminated more effectively than in the first and second embodiments.

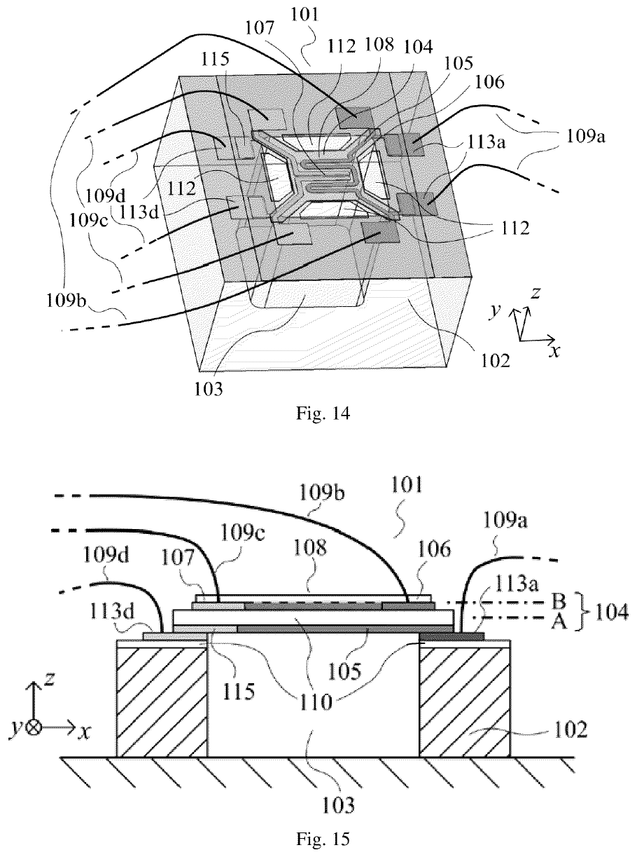

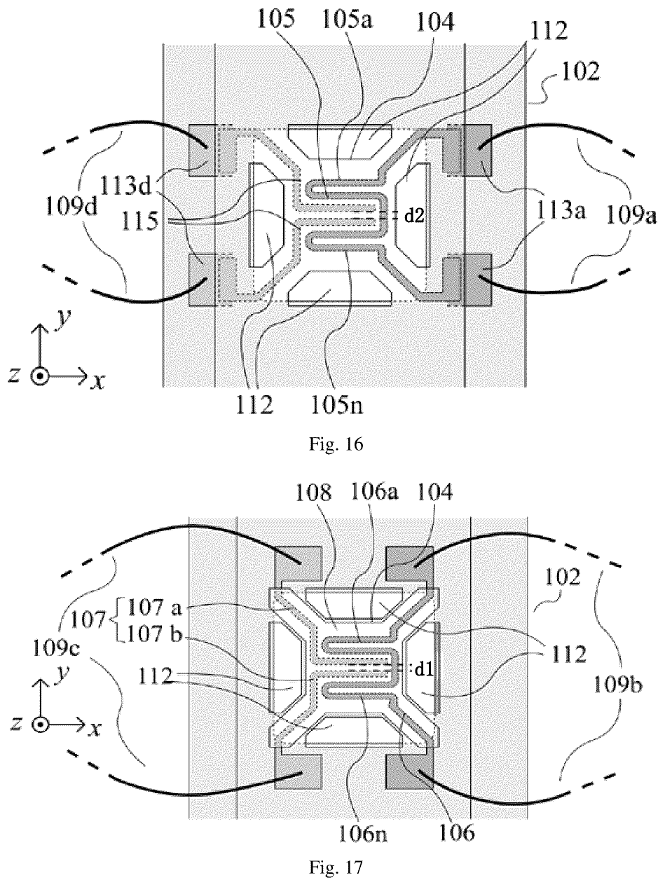

FIGS. 14-18 illustrate a third embodiment. FIG. 14 shows a structural schematic view according to the third embodiment of the disclosure. FIG. 15 shows a side view (in a direction along axis-y) according to the third embodiment. FIG. 16 shows a top view (in a direction along axis +z) taken along the plane A of FIG. 15. FIG. 17 shows a view in a direction (along +z) taken along the plane B of FIG. 15. It should be noted that the first heating resistor 105 on the lower side of the thin film 104 and the bonding wires 109a, the second thermistor electrode 115, and the bonding wires 109d are omitted in FIG. 17, facilitating a clear view of the arrangement of the upper surface of the thin film 104. The details will not be repeated here.

In the third embodiment, not only is a thermistor electrode 107 disposed on the surface where the second heating resistor 106 is disposed, but the second thermistor electrode 115 is also provided on the surface where the first heating resistor 105 is disposed. This differs from the first and second embodiments. In such configuration, it is advantageous that the straight-line portions at the beginning and the end of the meandering shape of the first heating resistor 105 and the straight-line portions at the beginning and the end of the meandering shape of the second heating resistor 106 are arranged at the top and bottom in an overlapped manner.

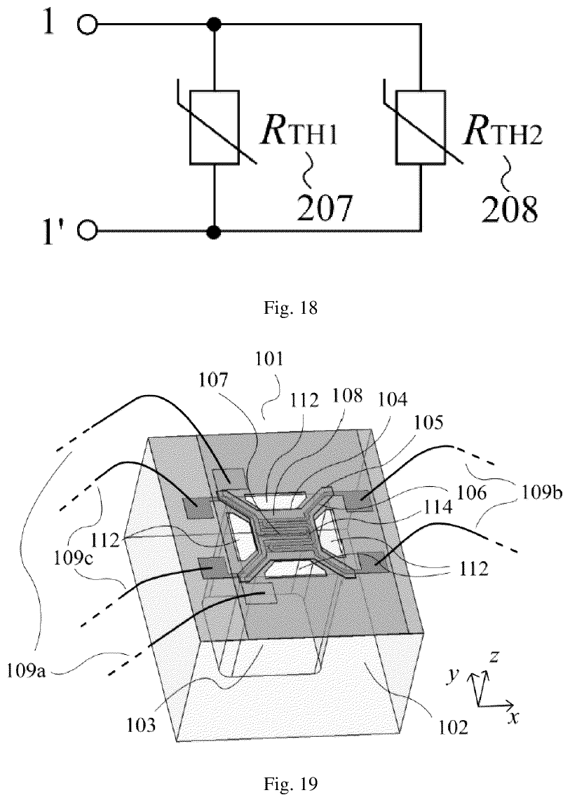

It is advantageous that the gaps d2 and d1 defined by the second thermistor electrode 115 and by the thermistor electrode 107 as shown in FIGS. 16 and 17 are aligned, and the lengths and pattern widths of the opposing electrodes which define these gaps are aligned. In such a case that the heating resistors and the thermistor electrode at the top and bottom of the film have the same configuration, the deformation of the thin film 104 in the Z-axis direction can be further reduced. FIG. 18 shows a schematic view of two thermistor electrodes according to the third embodiment, i.e., the wiring circuit diagram for the thermistor electrode 107 and the second thermistor electrode 115. This diagram illustrates the circuit in which the thermistor electrode 107 and the second thermistor electrode 115 are connected in parallel. In FIG. 18, RTH1 represents the resistance 207 of the thermistor electrode 107, and RTH2 represents the resistance 208 of the second thermistor electrode 115.

The composite resistance in the parallel state can be calculated by the formula (1) as follows.

K_{TH1}+K_{TH2}

For example, if the resistance values of RTH1 and RTH2 are the same, then the composite resistance R =RTH1/2=RTH2/2. In other words, the resistance value of the composite resistance R is less than that of RTH1 and also less than that of RTH2. It indicates that in the third embodiment, the distance of the gap (i.e., d1 in FIG. 17) of the thermistor electrode 107 and the distance of the gap (i.e., d2 in FIG. 16) of the second thermistor electrode 115 can be greater than the gap distances in the first and second embodiments, to obtain the same resistance values. If the distances of the gaps d1 and d2 are very small, the variations caused by etching accuracy will increase. Hence, increasing the gap distances as in the third embodiment can facilitate the improvement in manufacturing accuracy.

As described in the first and second embodiments above, the thermistor electrode 107 is in contact with the thermosensitive resistor material 108. However, the second thermistor electrode 115 may or may not be provided with the thermosensitive resistor material 108. By providing the thermosensitive resistor material 108, the surface where the thermistor electrode 107 is provided and the surface where the second thermistor electrode 115 is provided can have the same configuration, making the effect of canceling out the displacement in the Z-axis direction even more effective. Nevertheless, one skilled in the art should make a selection based on actual situations, because providing the thermosensitive resistor material 108 may make the manufacturing process more complicated.

### Fourth Embodiment

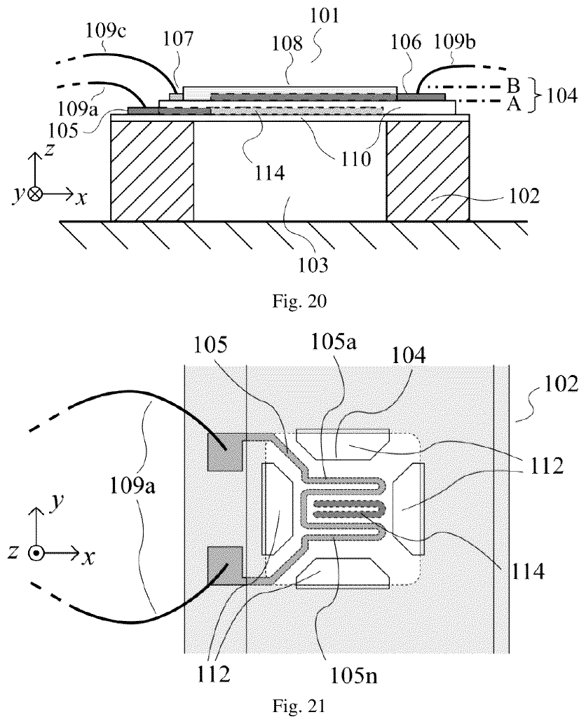

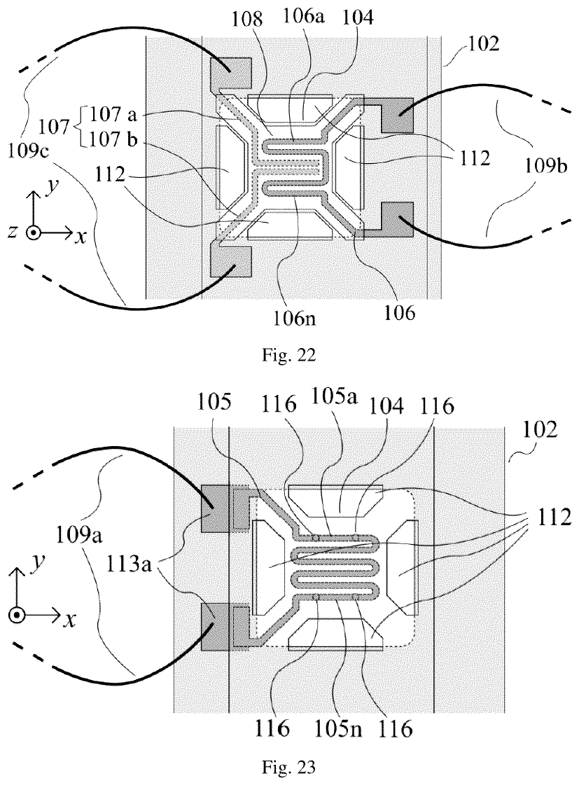

The third embodiment mentioned above describes the configuration which includes the second thermistor electrode 115, while the fourth embodiment uses a virtual pattern (dummy pattern), as shown in FIGS. 19-22, instead of the second thermistor electrode 115. In particular, FIG. 19 shows the structural schematic view according to the fourth embodiment. FIG. 20 shows a side view (in a direction along axis −y) according to the fourth embodiment. FIG. 21 shows a top view (in the direction along +z) taken along the plane A in FIG. 20. FIG. 22 shows a view taken along the plane B in FIG. 20, in the direction (along +z). It should be noted that, FIG. 22 shows a schematic view taken along the plane B, viewed from top, according to a fourth embodiment of the disclosure. Since it is viewed from top (a top view), the components such as the first heating resistor 105 provided on the lower side of the thin film 104 and the bonding wires 109a are not shown, thereby facilitating a clear view for the configuration of the upper side of the thin film 104.

As an example of the fourth embodiment, FIG. 21 shows a schematic view taken along the plane A, viewed from top, according to the fourth embodiment of the disclosure. Herein, FIG. 21 illustrates an example in which a U-shaped virtual pattern 114 is provided. If the virtual pattern 114 is not designed here, the thermistor electrode 107 itself will undergo the thermal expansion upon receiving the heat, thereby leading to the deformation of the thin film 104. Hence, in the fourth embodiment, the virtual pattern (dummy pattern) 114 is disposed on the side of the thin film 104 opposite to the thermistor electrode 107, on the layer (e.g., the layer where the first heating resistor 105 is disposed) which is overlapped with the thermistor electrode 107. In this way, the virtual pattern 114 can also receive the thermal flow from the heating resistors, and thus will undergo the thermal expansion in the direction opposite to that of the thermistors 107, thereby canceling out the deformation caused by the thermistor electrode 107. Consequently, the deformation of the thin film 104 can be inhibited. In FIG. 21, the virtual pattern 114 is shown in a U-shape. However, it is conceivable that it may have a U-shape in a direction rotated by 180° relative to the orientation in the figure. Alternatively, it doesn't necessarily need to have a U-shape, and it may have an unclosed O-shape or may be implemented by using two lines. By providing the virtual pattern 114, the deformation of the thin film 104 due to the deformation of the thermistor electrode 107 can be avoided.

### Fifth Embodiment

As explained in the first embodiment described above, if the thin film 104 is made of an extremely thin film, the thermal flow can be transferred to the other heating resistor through the thin film when the voltage is applied to either one of the first heating resistor 105 and the second heating resistor 106 to generate heat. Thus, the voltage is not necessarily to be applied to both heating resistors. Alternatively, to achieve the heat transfer effect, several thermal vias 116 are provided in the fifth embodiment, to facilitate the thermal coupling between the two heating resistors even more effectively.

Preferably, to increase the degree of thermal coupling between the straight lines at the beginning and the end of the meandering shape of the first heating resistor 105 and the straight lines at the beginning and the end of the meandering shape of the second heating resistor 106, thermal vias 116 may be provided for the thermal coupling therebetween. The presence of the thermal vias 116 makes it possible to achieve reliable heating for the two heating resistors by heating either one of the heating resistors. Due to the thermal vias 116, it can achieve thermal contact with the first heating resistor 105 and the second heating resistor 106. Even when they are not in direct contact, bringing them closer together can increase the degree of thermal coupling.

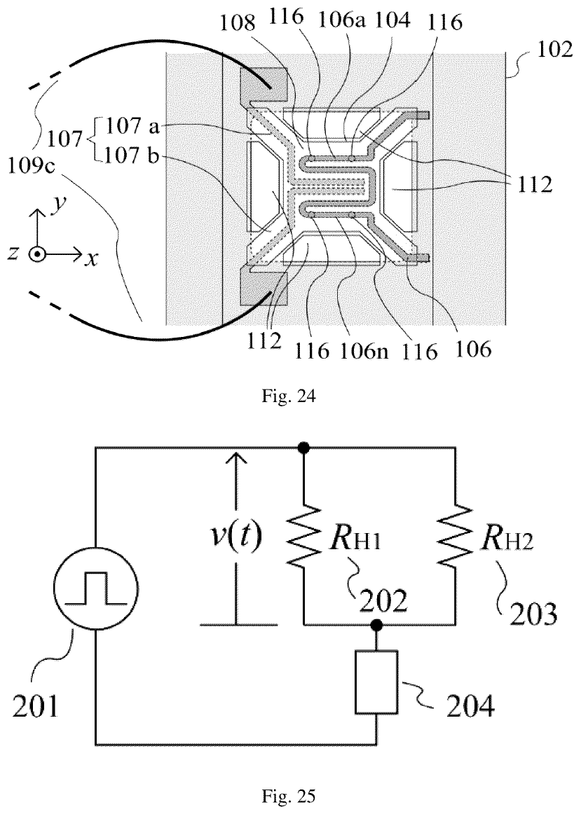

FIG. 23 shows a structural schematic view according to a fifth embodiment of the disclosure, in which the first heating resistor and the second heating resistor are provided with thermal vias. FIG. 23 and FIG. 24 illustrate an example of the configuration, in which the first heating resistor 105 and the second heating resistor 106 are in contact through the thermal vias 116. In particular, FIG. 23 shows a plane, which corresponds to FIG. 3 that illustrates the first embodiment, and depicts the pattern on the lower side of the thin film 104. In other words, it shows the first heating resistor 105, the connection pads 113a, the bonding wires 109a, and the thermal vias 116. FIG. 24 shows a plan view, which corresponds to FIG. 4 that illustrates the first embodiment, of the fifth embodiment. It depicts the pattern on the upper side of the thin film 104. In other words, it shows the second heating resistor 106, the thermistor electrode 107, and the thermal vias 116. In FIGS. 23 and 24, the straight lines at the beginning and the end of the meandering shape of the first heating resistor 105 and the straight lines at the beginning and the end of the meandering shape of the second heating resistor 106 are in contact with the two thermal vias 116, respectively. If the first heating resistor 105 and the second heating resistor 106 are in full electrical contact through the thermal vias 116, when applying the voltage to either the first heating resistor 105 or the second heating resistor 106, the current can flow to the other heating resistor, thereby generating Joule heat to generate heat. In the configurations as shown in FIGS. 23 and 24, as the voltage is only applied to the first heating resistor 105, the bonding wires on the side where the second heating resistor 106 is disposed can be omitted. The details will not be repeated here. In such configuration, the thermal expansions of the first heating resistor 105 and the second heating resistor 106, caused by Joule heating, can also cancel out the displacements on the thin film 104 caused by their respective heating resistors.

Furthermore, even when the first heating resistor 105 and the second heating resistor 106 merely get closer due to the thermal vias 116 rather than in full contact, the effect of thermal coupling can be improved. Thus, even when the voltage is applied to either the first heating resistor 105 or the second heating resistor 106, the two heating resistors can undergo the thermal expansion to cancel out the deformation of the thin film 104. Consequently, the voltage is not necessarily to be applied to both heating resistors.

The disclosure is described with reference to the first to fifth embodiments mentioned above. However, the disclosure may be implemented in various other forms of applications. For example, FIG. 5 shows a configuration, in which the first heating resistor 105 and the second heating resistor 106 are simply connected in parallel to the voltage source 201, on condition that the voltage is simultaneously applied to the two heating resistors to heat them. Other circuit elements such as resistive elements, inductive elements, capacitive elements, and semiconductor elements may be further connected in parallel to the voltage source 201. For example, referring to FIG. 25 which shows a schematic circuit diagram for driving the first heating resistor and the second heating resistor according to the disclosure, the circuit configuration of the series circuit 204 provided here can be selected based on actual needs.

Furthermore, the bridge circuit configuration, which is commonly used in sensor circuits, may be chosen. If voltages are simultaneously applied to the resistance 202 of the first heating resistor and the resistance 203 of the second heating resistor, the effect of canceling out deformations according to the disclosure can be achieved. A combination of incorporating the first heating resistor 105 and the second heating resistor 106 in a plurality of circuits may be chosen. Additionally, though the first heating resistor and the second heating resistor are defaulted to two layers, a configuration with more layers of the heating resistors shall fall within the scope of the present disclosure, as long as it can achieve the effect of canceling out each other. That is, “first”, “second” should not be construed as limitations on quantity.

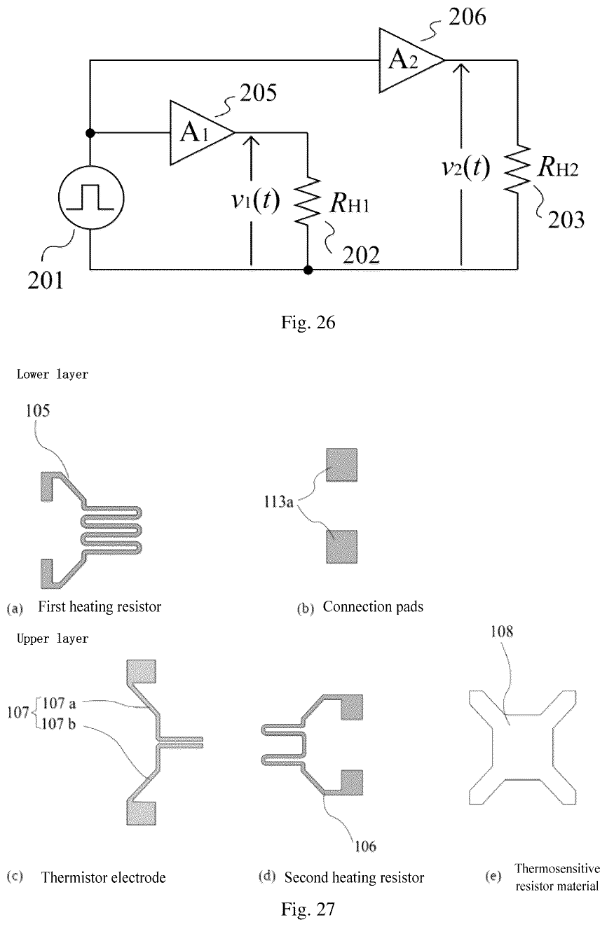

Though the voltage source 201 is depicted as a simple ON/OFF pulse source in FIG. 5, it may be configured to detect external air temperature, provide feedback and adjust the voltage level, switch the voltage level as needed, and so on. FIG. 26 illustrates a schematic diagram of a circuit with voltage amplifiers according to the disclosure. The circuit example in FIG. 26 discloses a circuit containing voltage amplifiers A1 and A2, which can provide different levels of voltage to the resistance 202 of the first heating resistor and the resistance 203 of the second heating resistor, respectively.

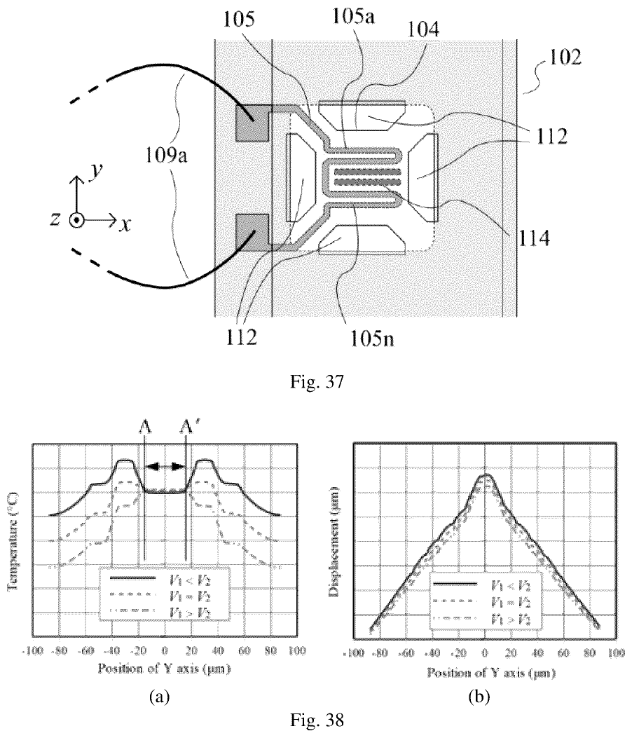

Furthermore, during the operation of the sensor, with respect to the voltages, the voltage applied to the first heating resistor 105 may be the first voltage V1, and the voltage applied to the second heating resistor 106 may be the second voltage V2, wherein the first voltage V1 may be greater than or equal to the second voltage V2. In particular, please refer to FIG. 38 which shows relevant curve graphs according to one of the embodiments of the disclosure. Herein, FIG. 38(a) on the left side shows a curve graph for illustrating the relationship between heats and positions, and FIG. 38(b) on the right side shows a curve graph for illustrating the relationship between displacements and positions. Each graph depicts three sets of data. Among the temperature distributions of these data, what counts is the temperature of the detection portion of the gas sensor. Thus, the voltages V1 and V2 are adjusted to keep the temperature between A and A′ in FIG. 38(a) constant. That is, in order to achieve the same temperature between A and A′, adjustments for the levels of the voltage V1 applied to the first heating resistor 105 and the voltage V2 applied to the second heating resistor 106 may be required, including three cases: V1<V2, V1=V2, and V1>V2.

As shown in FIG. 38(a), it can be seen that in the case of V1<V2, overheating is required near −30 micrometers and 30 micrometers, which may cause damage to the gas sensor, and may alter the properties of the thin film 104 in the long run. Besides, as shown in FIG. 38(b), it is confirmed that the displacement in the case of V1<V2 is greater than that under other conditions. In other words, in the case of V1=V2 or V1>V2, the corresponding constant temperature effect can be achieved by adjustments.

In the first and second embodiments, the first heating resistor 105 is disposed at the bottom of the thin film 104, while the second heating resistor 106 and the thermistor electrode 107 are disposed at the top of the thin film 104. Alternatively, the first heating resistor 105 may also be disposed on top of the thin film 104, and the second heating resistor 106 and the thermistor 107 may also be disposed at the bottom of the thin film 104. In such a case that the thermistor is positioned either at the top or bottom of the thin film 104, it can come into contact with incoming gases and function as a gas sensor.

Furthermore, the embodiments of the disclosure illustrate how the connection pads of the first heating resistor 105, the connection pads of the second heating resistor 106, the connection pads of the thermistor electrode 107, and the connection pads of the second thermistor electrode 115 are arranged to not overlap with one another and meanwhile to be respectively connected with bonding wires. The position of the portions of the thin film 104 where the connection pads are not disposed is not limited to those disclosed in the embodiment, because it has an insignificant effect on the displacement of the thin film 104 in the Z-axis direction.

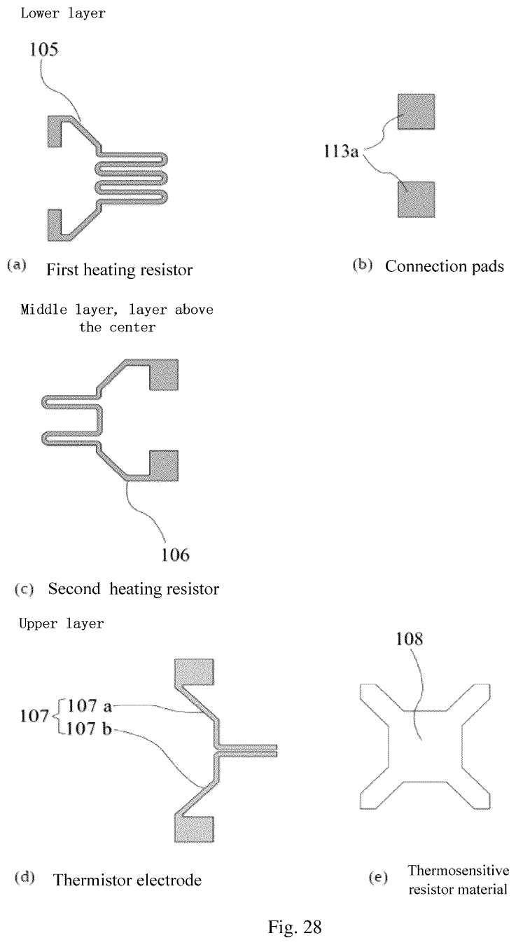

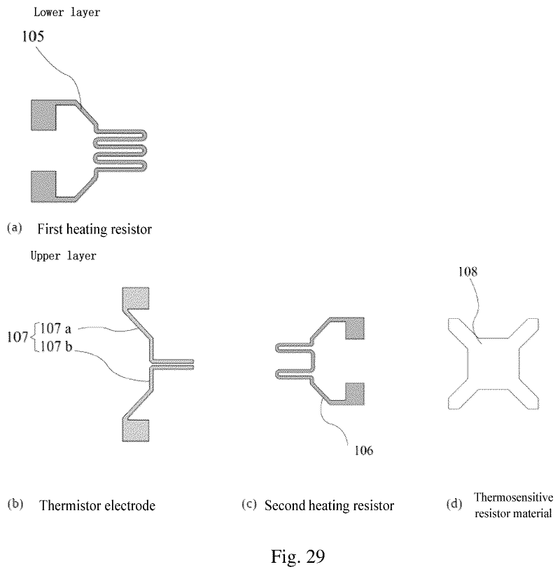

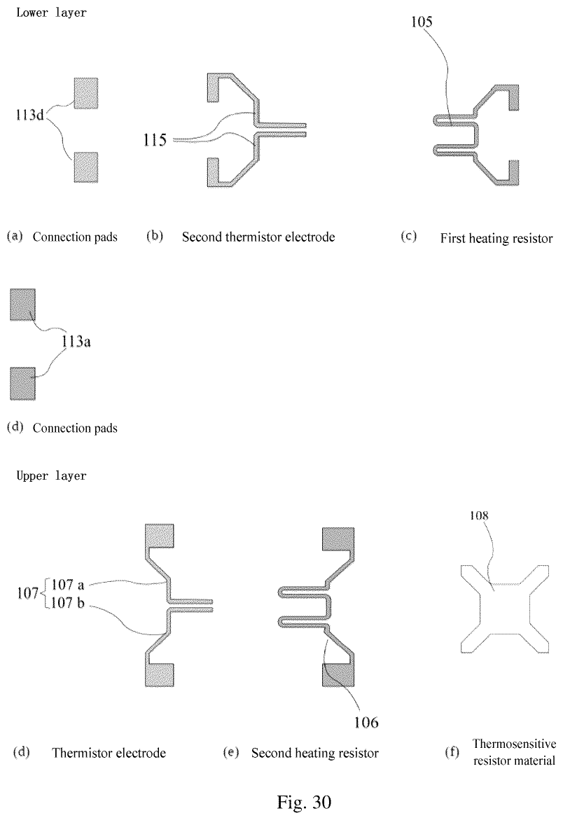

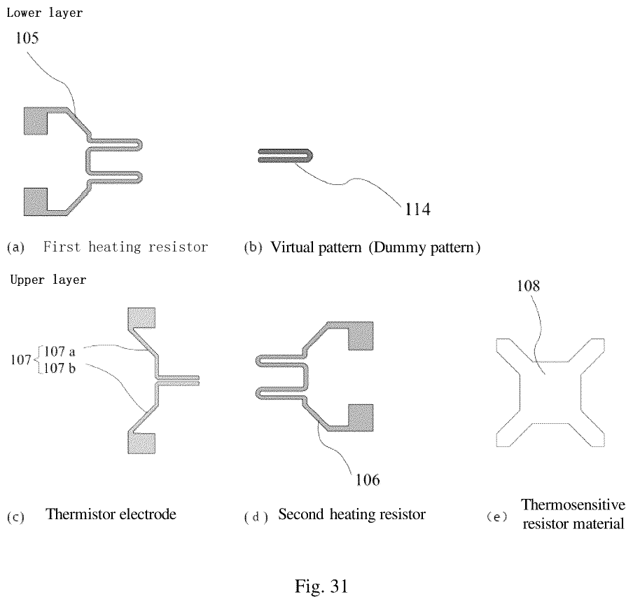

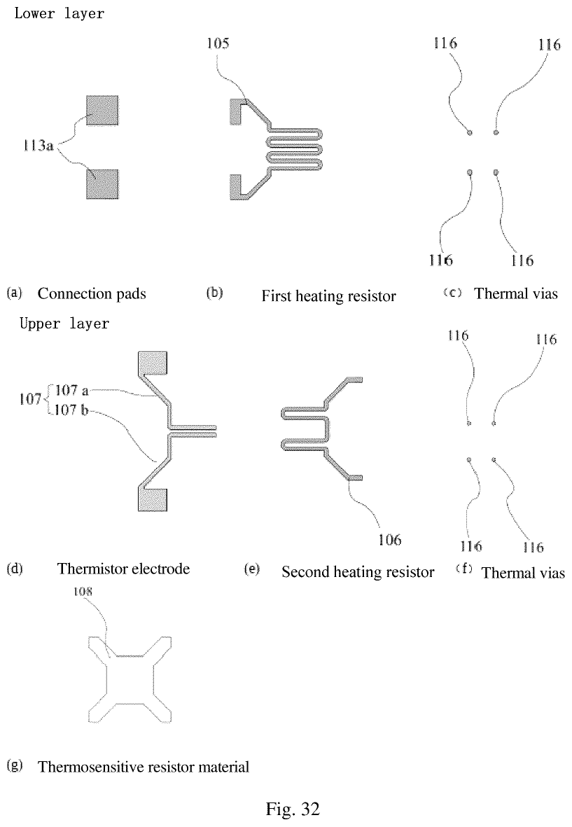

The first to fifth embodiments have been described above to illustrate the disclosure. To facilitate understanding, various implementations of the components of the disclosure have been illustrated. Please refer to FIGS. 27 to 32. FIG. 27 illustrates the first shape structural design of individual parts of the disclosure, in which the first heating resistor and the second heating resistor are respectively disposed on the upper and lower layers of the thin film, and the thermistor electrode and the second heating resistor are provided on the same plane. FIG. 28 illustrates the second shape structural design of individual parts of the disclosure, in which the thin film includes three layers. Herein, the first heating resistor is provided on the lower layer, i.e., on the side of the thin film near the cavity. The second heating resistor is provided on the middle layer. It may be provided on the middle layer or at a position above the center, depending on actual needs. The thermistor electrode is provided on the upper layer, with the thermosensitive resistor material at least partially covering the thermistor electrode. In other words, according to the second shape structural design, the first heating resistor, the second heating resistor, and the thermistor electrode are located on different layers. FIG. 29 illustrates the third shape structural design of individual parts of the disclosure, which differs from the first shape structural design of FIG. 27 in that it is designed without connection pads. FIG. 30 illustrates the fourth shape structural design of individual parts of the disclosure, in which the second heating resistor and the thermistor electrode are located on the upper layer, and the first heating resistor and the second thermistor electrode are located on the lower layer. FIG. 31 illustrates the fifth shape structural design of individual parts of the disclosure, which introduces the virtual pattern (dummy pattern). Herein, the first heating resistor and the virtual pattern are provided on the lower layer, and the second heating resistor and the thermistor electrode are located on the upper layer. FIG. 32 illustrates the sixth shape structural design of individual parts of the disclosure, which introduces the thermal vias. Herein, the first heating resistor and corresponding thermal vias are provided on the lower layer, and the second heating resistor, the thermistor electrode and corresponding thermal vias are located on the upper layer. FIGS. 27 to 32 respectively illustrates different structural designs for core components such as the first heating resistor, the second heating resistor, the thermistor electrode, the dummy pattern (virtual pattern), the thermal vias, and so on. Based on actual design requirements, those skilled in the art can make a selection which are not limited to the structural designs described in the first to fifth embodiments or FIGS. 27 to 32. The details will not be repeated here.

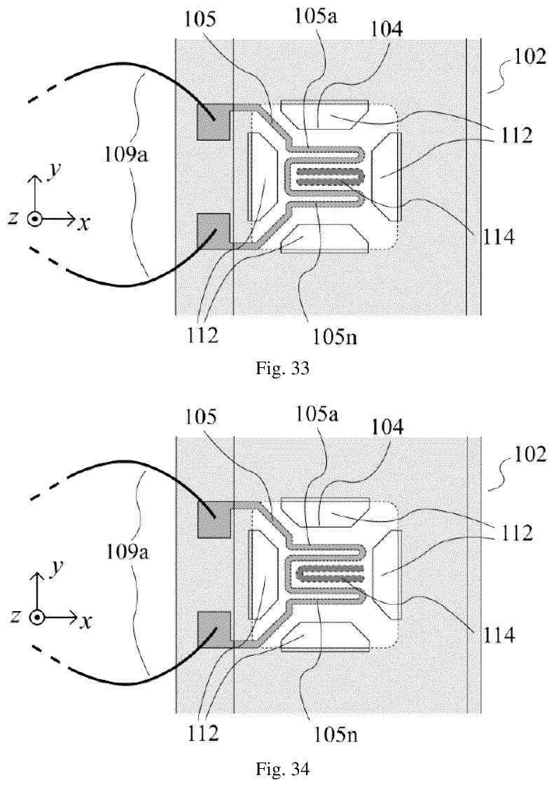

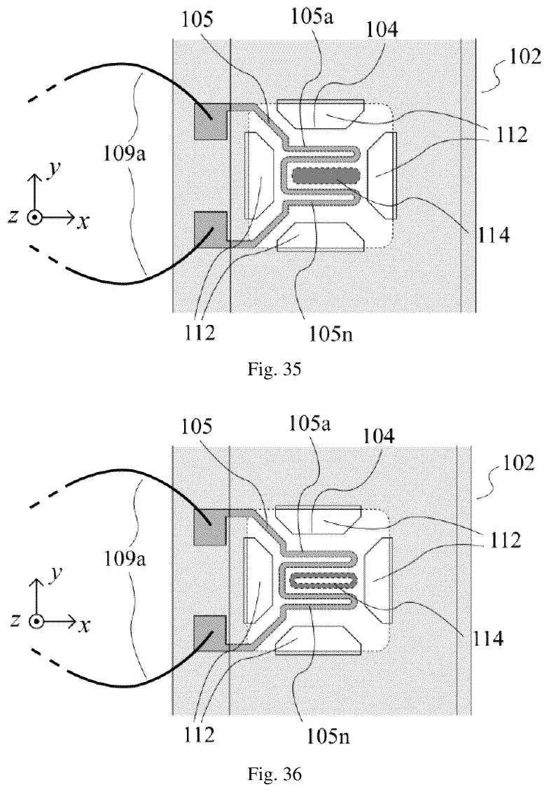

For the same reason, to facilitate understanding, various structural design implementations for the dummy pattern (virtual pattern) of the disclosure have been further illustrated. Please refer to FIGS. 33-37. FIG. 33 shows a schematic plane view of the disclosure, provided with a first virtual pattern structure. FIG. 34 is a schematic plane view of the disclosure, provided with a second virtual pattern structure. FIG. 35 is a schematic plane view of the disclosure, provided with a third virtual pattern structure. FIG. 36 is a schematic plane view of the disclosure, provided with a fourth virtual pattern structure. FIG. 37 is a schematic plane view of the disclosure, provided with a fifth virtual pattern structure. As can be seen from FIGS. 33 to 37 mentioned above, the dummy pattern (virtual pattern) may be designed in various shapes such as U-shape, surface-shape, O-shape, or line-shape. Apparently, it is not limited to the structural designs as shown in FIGS. 33 to 37. The details will not be repeated here.

In another embodiment, the disclosure provides a gas sensor comprising the sensor element as mentioned above. According to the disclosure, a plurality of sensor element 101 may be produced, which may be assembled into a gas sensor. According to the disclosure, the plurality of sensor elements 101 may be provided on one single substrate 102. As the plurality of sensor elements 101 may be provided on one single substrate 102, the sensor elements 101 may be arranged adjacent to each other, whereby the mounting area can be reduced. There are no specific limitations here.

The sensor element and the gas sensor provided in the embodiments of the disclosure have advantages including at least one of the following.

The first heating resistor and the second heating resistor can cooperate with each other. The thermal stress in the thickness direction of the film caused by the heat generated by the first heating resistor can be opposite in direction to the thermal stress in the thickness direction of the film caused by the heat generated by the second heating resistor. This can allow the stresses to cancel out each other, and thus cancel out the thermal stress in the thickness direction of the film. In such a case, the thermal stress in the thickness direction of the film can be significantly reduced, thereby effectively avoiding deformation of the film structure, stabilizing the resistance characteristics within the gas sensor, and making them less prone to change. Consequently, the stable performance of the gas sensor can be ensured.

All the above embodiments merely describe some implementations of the present disclosure, which are illustrated in detail in a relatively specific manner. However, they are not intended to limit the scope of the present disclosure. It should be noted that those skilled in the art may obtain various equivalents and modifications included in the scope of the present disclosure, without departing from the technical concept of the present disclosure. Hence, the invention is defined by the appended claims.

1. A sensor element, characterized by comprising a substrate and a thin film arranged on the substrate; wherein:
the substrate is provided with a cavity and an opening which is in communication with the cavity;
the thin film is supported on the opening and partially covers the cavity;
the thin film comprises a first heating resistor and a second heating resistor, each of the first heating resistor and the second heating resistor is shaped in a meander line, the first heating resistor is positioned on a side of the thin film near the cavity, and the second heating resistor is positioned on a side of the thin film away from the cavity; and
a linear segment at an end portion of the first heating resistor is at least partially overlapped with a linear segment at an end portion of the second heating resistor to allow a thermal stress induced by the first heating resistor and a thermal stress induced by the second heating resistor to cancel out each other.

2. The sensor element according to claim 1, wherein the thin film further comprises a thermistor electrode and a thermosensitive resistor material; and
the thermistor electrode lies on a same plane as either the first heating resistor or the second heating resistor, and the thermosensitive resistor material at least partially covers the thermistor electrode.

3. The sensor element according to claim 1, wherein the thin film further comprises a thermistor electrode and a thermosensitive resistor material;
the first heating resistor is positioned on the side of the thin film near the cavity;
the second heating resistor is positioned on a side of the thin film away from the first heating resistor; and
the thermistor electrode is positioned on a side of the thin film away from the second heating resistor, and the thermosensitive resistor material at least partially covers the thermistor electrode.

4. The sensor element according to claim 1, wherein the thin film further comprises a first thermistor electrode, a first thermosensitive resistor material and a second thermistor electrode; and
the first thermistor electrode lies on a same plane as the first heating resistor, the first thermosensitive resistor material at least partially covers the first thermistor electrode, and the second thermistor electrode lies on a same plane as the second heating resistor.

5. The sensor element according to claim 4, wherein the thin film further comprises a second thermosensitive resistor material, and the second thermosensitive resistor material at least partially covers the second thermistor electrode.

6. The sensor element according to claim 2, wherein the thermistor electrode is disposed inside a non-linear portion of the first heating resistor or of the second heating resistor.

7. The sensor element according to claim 1, wherein a non-linear portion of the first heating resistor is curved in a direction rotated 180° relative to a direction in which a non-linear portion of the second heating resistor is curved.

8. The sensor element according to claim 1, wherein the first heating resistor and the second heating resistor are connected in parallel in a circuit.

9. The sensor element according to claim 8, wherein the circuit is further provided with a voltage amplifier for adjusting a voltage applied to the first heating resistor and the second heating resistor.

10. The sensor element according to claim 1, wherein the thin film further comprises an insulator provided between the first heating resistor and the second heating resistor, and the first heating resistor and the second heating resistor are isolated from each other by the insulator.

11. The sensor element according to claim 2, wherein the thin film further comprises a first insulator;
the thermistor electrode lies on a same plane as the second heating resistor, and the thermosensitive resistor material at least partially covers the thermistor electrode and the second heating resistor;
the first insulator is positioned on a layer that is lower than the second heating resistor; and the first insulator partially covers the first heating resistor, and the first heating resistor is positioned on a side of the first insulator away from the second heating resistor.

12. The sensor element according to claim 11, wherein the sensor element further comprises a second insulator; and
The second insulator is positioned on a layer that is lower than the first heating resistor.

13. The sensor element according to claim 2, wherein the thin film is provided with a dummy pattern for receiving heat energy to expand, to allow the deformation caused by the thermistor electrode to be canceled out.

14. The sensor element according to claim 1, wherein the sensor element is provided with one or more thermal vias for allowing the first heating resistor and the second heating resistor to achieve thermal coupling.

15. The sensor element according to claim 1, wherein, during operation of the sensor, a voltage applied to the first heating resistor is a first voltage, a voltage applied to the second heating resistor is a second voltage, and the first voltage is greater than or equal to the second voltage.

16. A gas sensor, characterized by comprising the sensor element according to claim 1.
